(19) United States
(12) Patent Application Publication
(10) Pub. No.: US 2026/0294578 A1
(43) Pub. Date: Oct. 1, 2026

(54) Electromedical device and measuring kit

(71) Applicant: X-PRESSURE, TOULOUSE (FR)

(72) Inventor: Pierre-Edouard DE LAMY, Toulouse (FR)

(21) Appl. No.: 19/252,331

(22) Filed: Jun. 27, 2025

(30) Foreign Application Priority Data
Jun. 27, 2024 (FR) 2406979

(51) Int. Cl. A61B 90/00; A61B 5/00

(52) U.S. Cl. CPC A61B 90/03; A61B 5/7225; A61B 2560/0214; A61B 2562/227

(57) ABSTRACT

“An electro-medical device (10) configured to relay, to a remote terminal, an analog signal representing the physiological data of a patient such as the intracranial pressure or the blood pressure of the patient.

The electro-medical device (10) includes
a rechargeable battery (14),
a first electronic connector (30) which has a first axis of connection A-A, making it possible to receive the incoming analog signal, and
a power supply connector (60) which is connected electrically to the battery (14), the power supply connector (60) comprising a second axis of connection B-B.

The spatial proximity between the two connectors (30, 60) and the intersection of their axes of connection A-A, B-B prevent the first electronic connector (30) and the power supply connector (60) from being coupled at the same time.”

### TECHNICAL FIELD

The invention relates to an electro-medical device configured to relay, to a remote terminal, an analog signal representing the physiological data of a patient. For example, the physiological data may correspond to the intracranial pressure or to the blood pressure of the patient, etc. The invention may also relate to a measurement kit for measuring physiological data which comprises an electro-medical device according to the invention and a physiological data sensor.

### PRIOR ART

Currently, the development of an electro-medical device, such as a portable electric device for relaying physiological data of a patient between a sensor and a remote terminal (smartphone, computer, etc.), is subject to various regulations which impose technical constraints. Most of the time, these technical constraints may be prescribed for safety reasons linked to biocompatibility, sterilization, the age of the device, as well as physical protection of the patient against electrical hazards or electroacoustic hazards (Radio, EMF). The cybersecurity of medical data is another important aspect that has to be taken into account in the industrial production of new medical devices which are capable of connecting to local or public networks.

As regards portable electric devices for medical use, the regulatory requirements notably include the obligation for the manufacturer to ensure that the high voltage alternating current, coming from a wall outlet for example, cannot be passed on to the patient since this would pose a risk of death. Regulations require in particular that the electro-medical device must make it possible to avoid short-circuits and prevent any high voltage electricity from passing from a high voltage current source to the patient. Most of the time, the high voltage current source consists of a wall outlet into which the electro-medical device is plugged to charge its battery or to operate. Reverse polarity, limitation of the output voltage as well as protection against overheating of the electro-medical device are also issues that must be addressed.

In order to protect patients against electrical hazards, some manufacturers fit the medical portable electric devices with electronic components such as AC/DC or DC/DC converters, or various systems for isolation/protection of the device, which are included on the circuit board. However, electronic components such as these are very expensive as well as very bulky because they must have robust dimensions in order to protect the patient. It is thus difficult for a manufacturer to produce, at low cost, devices that are compact, and portable.

To solve this problem while keeping production costs under control, other manufacturers have chosen to fit their medical portable electric devices with a removable rechargeable battery without providing a power supply connector on the devices to ensure that the battery cannot be charged from the power grid while it is connected to the device. Manufacturers therefore have to provide specific designs in terms of connector technology to prevent the battery from being charged while it is connected to the device. The aim is thus to force the user to remove the rechargeable battery from the device in order to charge it. The battery must thus be taken out of the device in order to be charged. Although this tackles the problem of safety, a removable battery has a higher chance of being lost than a non-removable internal battery. This is especially the case in hospital environments where equipment is used by several practitioners, or indeed by several teams of practitioners. Moreover, from a design point of view, this option requires that an opening and a lid be provided on the casing so that the battery can be taken out for charging and then put back into the casing, as applicable. This lid must in some cases be provided with a seal to prevent fluids from getting in and damaging the battery while it is in its housing. Such a solution is therefore unsatisfactory on many levels.

The invention aims to overcome all or some of these drawbacks.

### DISCLOSURE OF THE INVENTION

The invention aims to protect the patient from all types of electrical hazard posed by an electro-medical device, while keeping manufacturing costs under control.

To this end, the invention relates to an electro-medical device configured to relay, to a remote terminal, an analog electrical signal representing the physiological data of a patient such as the intracranial pressure or the blood pressure of the patient, the analog electrical signal being produced by an electronic physiological sensor, the electro-medical device comprising a casing which includes:
a rechargeable battery arranged in an internal space in the casing and supplying electrical power to the electro-medical device,
a first electronic connector which has a first axis of connection A-A, the first axis of connection A-A making it possible to connect the first electronic connector being configured, on the one hand, to be coupled to a connector fitting linked by means of a cable to the electronic physiological sensor, and on the other hand, to receive the incoming analog electrical signal, and
a power supply connector which is connected electrically to the battery and configured to be connected to a power supply connector fitting in order to transmit an electric current to the battery with a view to charging same, the power supply connector comprising a second axis of connection B-B,
   the first electronic connector and the power supply connector are, on the one hand, arranged spatially contiguously in a connection zone of the casing, and on the other hand, oriented spatially such that the first axis of connection A-A crosses the second axis of connection B-B, the intersection of the first axis of connection A-A and the second axis of connection B-B and the spatial proximity of the connectors preventing the first electronic connector and the power supply connector from being coupled at the same time.

The invention thus relates to a portable electric device for medical use for collecting and transmitting medical data, also referred to as physiological data. When the electro-medical device receives physiological data, it is indirectly connected to the patient by means of the sensor which produces and transmits the physiological data. For example, to measure the intracranial pressure of a patient, the sensor used is connected directly to the patient by means of a lumbar puncture, the sensor being connected to the lumbar puncture needle. Preventing the two connectors from being coupled at the same time makes it possible to prevent the electro-medical device from being connected simultaneously to the patient and to a source of high voltage alternating current. The risk of serious electrical incidents that could result in injury to the patient are thus limited.

The mechanical impediment is in particular due to the position of the power supply connector relative to the first electronic connector. More specifically, their spatial proximity and the intersection of their axes of connection are two factors which contribute to forming a mechanical impediment when one of the two connectors is coupled to an external complementary connector fitting which is itself connected by means of a cable to an electro-medical device. The first connector is thus configured to be coupled to a complementary connector fitting. The complementary connector fitting may be a male connector when the first connector is a female connector. The problem of electrical safety is thus dealt with by a particular mechanical design of the connection zone of the casing. The inventor thus addresses the technical problem without the use of expensive and bulky electronic components.

The axis of connection of a connector corresponds to the axis along which the complementary connector fitting or plug of the connector is plugged into the connector of the electro-medical device. The plug or the connector fitting is connected to an external element, such as a physiological sensor or a transformer, the latter being itself connected to the power grid via a power outlet.

Note that other types of data such as intracranial pressure, blood pressure, bladder pressure, fascial compartment pressure of the patient may also be relayed by an electro-medical device according to an embodiment of the invention.

In some embodiments, the distance between the power supply connector and the first electronic connector may be less than 5 cm, preferably less than 3 cm and more preferably less than 2 cm or even less than 1 cm. The spatial proximity combined with an inclination of at least one of the two connectors result in an intersection of the axes of connection and thus give rise to the mechanical impediment that prevents the two connectors from being coupled at the same time.

In order to allow the connection of a maximum of physiological data sensors, the first electronic connector may be selected from data connectors such as ODU MINI-MED®, LEMO®, Fisher®, Smith®, Attend®, jack, etc. Moreover, the first electronic connector may advantageously be a female connector. This makes it possible to provide a casing without any protruding parts, which is thus easier to transport, and may for example be carried in the pocket of a white coat. For the same advantageous reasons, the power supply connector may be a female connector. Moreover, the power supply connector may be selected from connectors such as USB-C, USB-A, DIN, etc. Preferably, the power supply connector is a USB-C connector.

Moreover, the rechargeable battery is preferentially not removable from the casing. For example, the casing may comprise a specific housing containing the battery permanently. This housing may notably be sealed so as to prevent the battery from being removed. This makes it possible to address the issue of electrical safety while keeping down the costs of manufacturing the casing. To be specific, the fact that the battery is not removable means that the manufacturer does not have to provide an opening in the casing, which limits the number of parts and simplifies the design of the casing of the electro-medical device.

In some embodiments, the first electronic connector and the power supply connector may be formed in a niche that is set back or recessed relative to a wall of the casing, the niche then forming the connection zone of the casing. Since the niche is set back from the wall of the casing, it helps to make the connection zone mechanically congested, notably limiting access to the connectors.

In some embodiments, the first electronic connector is integrated in a first recessed plane that is recessed relative to a wall of the casing, the power supply connector being integrated in a second recessed plane that is recessed relative to said wall, the first recessed plane and the second recessed plane crossing one another so that the axes of connection A-A and B-B intersect. The particular spatial orientation of the recessed planes helps to create spatial congestion in the connection zone which will then give rise to a mechanical impediment in collaboration, for example, with the male plug of a power supply connector fitting which is already plugged into the power supply connector. The connection zone then no longer has the space necessary to simultaneously couple the first electronic connector. Furthermore, the two recessed planes arranged in the outer walls of the casing make it possible to simplify the design of the casing and make it more compact, notably by reducing the number of parts of the casing. Conversely, in a flat wall, the inclined arrangement of the sensors involves the use of adapters at the wall and/or retention structures integrated in the casing to hold the connectors in the desired position. However, these internal structures take up space inside the casing which is no longer available for the circuit board of the casing. In such a case, it is then necessary to increase the volume of the casing to contain the internal positioning structures and the circuit board. It is obviously not desirable to increase the volume of the casing, as this goes against one of the aims of the invention, which is to offer a solution which is compact and flexible and which can be easily implemented in a hospital environment.

In particular, the first recessed plane and the second recessed plane may be arranged respectively on a first wall and a second wall of the casing which are adjacent and form an angle β with a value of less than 160°. Preferably, the value of the angle is between 60 and 150°, more preferably, the value of the angle is between 80° and 140°. The inclination of one wall relative to the other makes it possible to limit access to the connection zone as well as to cause the axes of connection of the two connectors to intersect. The first wall and the second wall may thus be arranged in such a way as to form a connection zone with an L shape or a half U shape. The axes of connection A-A and B-B respectively block by their intersection and prevent a data connector and the power supply connector from being coupled at the same time.

In some embodiments, the second recessed plane may create a break with the plane of said wall of the casing, the first recessed plane being set back from the plane of said wall of the casing and extending parallel to said wall of the casing, the second recessed plane for its part forming the link between said wall of the casing and the first recessed plane. Such a configuration makes it possible to give the connection zone a niche configuration. Advantageously, the wall of the casing which includes the connection zone is a lateral wall of the casing.

In some embodiments, the connection zone may advantageously be arranged in the vicinity of a corner of the casing. The position in the vicinity of a corner of the casing makes it possible to use the edges of the casing to limit access to the connection zone.

In some embodiments, the connection zone may be arranged on a wall of the casing which includes a protruding rim, said wall including a flat area that is set back from the rim. The protruding rim extends on the periphery of said wall of the casing. This rim helps to limit access to the connectors. In particular, the first recessed plane and the second recessed plane form with the protruding rim a niche set back from the flat area. The protruding rim accentuates the depth of the niche and limits access to the connectors somewhat further.

In some embodiments, the first recessed plane and the second recessed may form a shoulder in a wall of the casing.

In some embodiments, the first electronic connector may include a tube the annular end of which is arranged in a plane which is raised relative to the power supply connector, the power supply connector being inclined relative to the tube. Such a configuration makes it possible to cause the axes of connection to intersect while reducing the distance between the two connectors.

In particular, the first axis of connection A-A and the second axis of connection B-B may form an angle α the value of which is less than 150°, preferably, the value of the angle α is less than 145°, and more preferably, the value of the angle α is less than 140°. The value of the angle α may also be less than 120° and preferably, less than 100°. The value of the angle α may also be greater than 20°, preferably, the value of the angle α is greater than 30°, more preferably the value of the angle α is greater than or equal to 40°. The value of the angle α may thus be greater than 50°, and preferably greater than 60°. Such angular values associated with a spatial proximity of the two connectors make it possible to create a mechanical impediment preventing the coupling of a second connector while the first connector is coupled to a connector fitting external to the electro-medical device.

In some embodiments, the electro-medical device may include a second electronic connector contiguous with the power supply connector, the second electronic connector comprising a third axis of connection C-C, the third axis of connection C-C crossing the axis of connection B-B of the power supply connector, the spatial proximity between the power supply connector and the third electronic connector and the intersection of their axes of connection B-B, C-C preventing the second electronic connector and the power supply connector from being coupled at the same time. Thus, the electro-medical device may include two electronic connectors configured to transmit data, the two electronic connectors possibly being connected simultaneously. The two connectors may be of the same type or of different types. The second electronic connector may be selected from data connectors such as ODU MINI-MED®, LEMO®, Fisher®, Smith®, Attend®, jack, etc. The second electronic connector is advantageously of a different type to the first electronic connector. For example, the second electronic connector may be a jack connector whereas the first is a LEMO® connector.

In all cases, these two electronic connectors make it possible to perform complex medical analyses which require, for example, synchronous acquisition of two types of physiological data such as an electroencephalography and the intracranial pressure of the patient. For the same reasons as given above, the second electronic connector may advantageously be a female connector.

So that the two electronic connectors can be connected simultaneously, the second electronic connector may be formed in the same plane as the first electronic connector. Furthermore, the second electronic connector may be arranged adjacent to the first electronic connector.

In some embodiments, the electro-medical device may include:
electronic means comprising a digital converter configured to convert the incoming analog electrical signal into a digital signal, and
a remote transmitter/receiver configured to transmit the digital signal to a remote terminal.

In some embodiments, the electronic means may include an internal memory configured to store the converted digital signal. It is thus possible to store the incoming signal even when the electro-medical device is not connected to a remote terminal at the time of acquisition of the physiological data.

In some embodiments, the electronic means may include an accelerometer configured to switch on the casing and/or pair the transmitter/receiver with a remote terminal. For example, two taps on a panel wall of the casing may trigger the process of pairing of the electro-medical device with a remote terminal. Another function that could be envisaged is the electro-medical device being switched on when it is subjected to a shaking motion above a predetermined amplitude or acceleration threshold.

In some embodiments, the electro-medical device may include a man/machine interface provided on a wall of the casing, the man/machine interface comprising one or more buttons, and/or one or more light indicators.

In some embodiments, the invention may relate to a measurement kit for measuring physiological data of a patient such as the intracranial pressure or the blood pressure of a patient, the measurement kit comprising an electro-medical device according to an embodiment of the invention and a physiological sensor configured, on the one hand, to measure the pressure of a physiological fluid of a patient, and on the other hand, to produce an analog electrical signal representative of said pressure. In particular, the electro-medical device is advantageously associated with an intracranial pressure sensor which is connected to the cerebrospinal fluid, the latter being made accessible by means of a lumbar puncture.

### DESCRIPTION OF THE DRAWINGS

Further features and advantages of the invention will become clearer from reading the following description, which is purely illustrative and must be read in conjunction with the appended drawings in which:

FIG. 1 is a perspective view of an electro-medical device according to an embodiment of the invention.

FIG. 2 depicts the casing of the device in FIG. 1, the connectors not being assembled.

FIG. 3 depicts an electro-medical device according to an embodiment of the invention, a first electronic connector of the casing being coupled to the male connector fitting of an electronic cable, the configuration of the casing and the male connector fitting preventing the power supply connector from being coupled.

FIG. 4 depicts an electro-medical device according to an embodiment of the invention, the power supply connector is coupled to a male connector fitting of a power supply cable, the configuration of the casing and the male connector fitting preventing the electronic connector from being coupled at the same time.

FIG. 5 depicts the connection zone of an electro-medical device according to an embodiment of the invention.

FIG. 6 depicts the connection zone of an electro-medical device according to another embodiment of the invention.

FIG. 7 depicts the electro-medical device of FIG. 6, a first electronic connector of the casing being coupled to the male connector fitting of an electronic cable, the configuration of the casing and the male connector fitting preventing the power supply connector from being coupled.

FIG. 8 depicts a system for transmitting the physiological data of a patient using an electro-medical device according to the invention.

FIG. 9 schematically depicts a cross section through the connection zone of an electro-medical device according to an embodiment of the invention.

### DESCRIPTION OF THE EMBODIMENTS

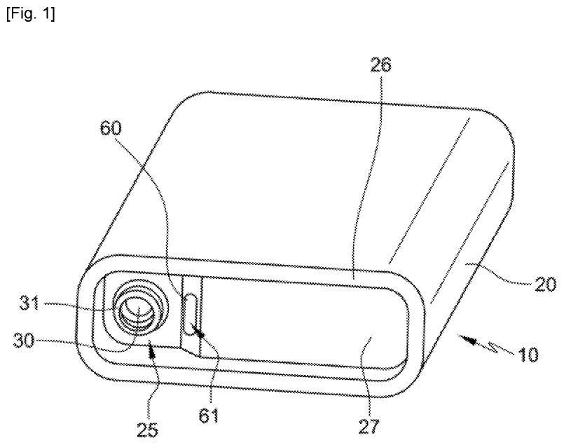

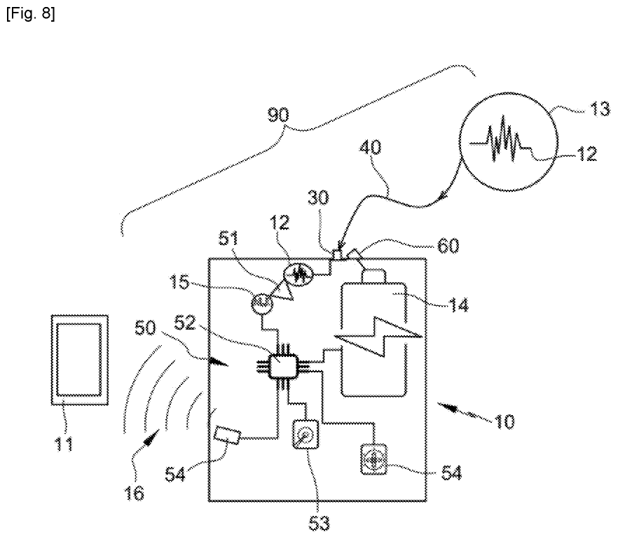

With reference to FIGS. 1 to 9, the invention relates to a medical electro-medical device 10. The electro-medical device 10 is in particular configured to relay physiological data of a patient to a remote terminal 11 as shown in FIG. 8. The remote terminal 11 may be a smartphone, a digital tablet, a computer or any other data processing device which has connectivity functions, preferably wireless, and a man machine interface making it possible to access the data relayed by the electro-medical device 10.

The physiological data may be in particular relayed in the form of an electrical signal 12 produced by a physiological data sensor 13. Most of the time, the sensors 13 used produce an analog electrical signal corresponding to the physiological data of a patient that they measure. For example, the inventors have described a sensor making it possible to measure the intracranial pressure of a patient by means of the pressure of the cerebrospinal fluid (CSF) as described in document WO2023/186817. The sensor is thus connected to a lumbar puncture needle so as to be placed in contact with the CSF. The pressure of the CSF is measured by means of a controlled volume of air which is interposed between a piezoelectric cell of the sensor and the CSF. The piezoelectric cell then retranscribes the pressure of the CSF, producing an analog electrical signal. Note that other types of data such as blood pressure, bladder pressure, fascial compartment pressure of the patient may also be relayed by the electro-medical device 10 in accordance with embodiments of the invention. The physiological sensor 13 is thus more specifically an electronic sensor. The analog electrical signal produced by this type of sensor 13 is a signal of predetermined duration which may comprise several components. For example, in the case of the measurement of intracranial pressure, the analog electrical signal produced by the sensor comprises: intracranial dynamics, the cardiac signal, the respiratory signal and probably B waves which are associated with vasomotor cycles.

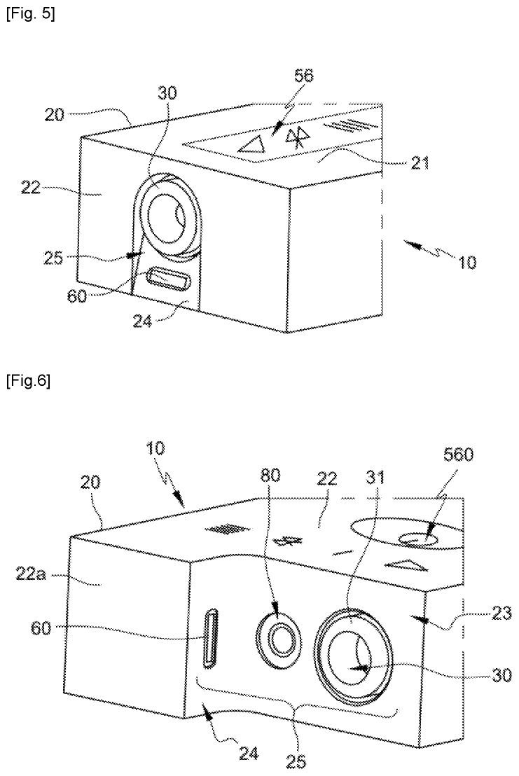

As shown in FIGS. 1 to 7, the electro-medical device 10 includes a casing 20 which has a substantially quadrangular geometric shape. The casing 20 thus has two panel walls 21 opposite one another and lateral walls 22 which close the casing and extend at the perimeter of the panel walls 21. The term “substantially” means that the edges and the corners of the quadrangular shape may be rounded as can be seen in FIGS. 5 to 7. Furthermore, in the embodiment of FIGS. 5 and 6, one lateral wall 22a has a recess. However, the casing may have any shape as long as it comprises a connection zone according to an embodiment of the invention.

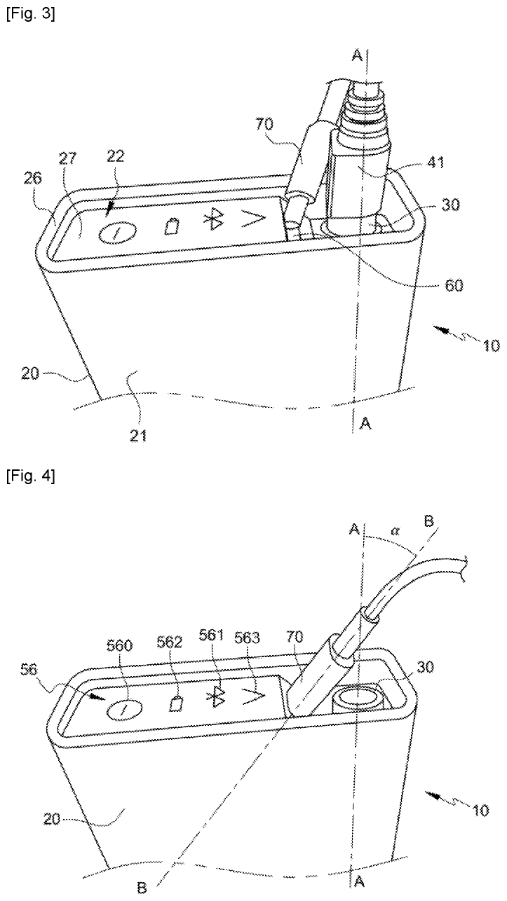

Moreover, the casing 20 comprises, in this connection zone, a first electronic connector 30. In this case, the first connector 30 is a female connector, and allows a data cable 40 to be plugged in with the aid of a male connector fitting 41. This is shown in particular in FIGS. 3, 7 and 8. The flow of data entering the electro-medical device 10 is in particular depicted in FIG. 8 by arrows which are shown on the cable 40.

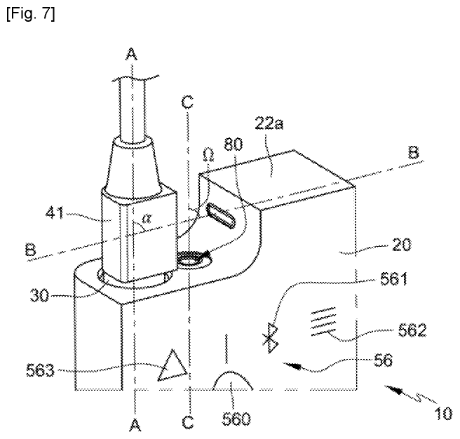

According to an embodiment illustrated notably in FIGS. 4 and 7, the first connector 30 has a first axis of connection A-A. The axis of connection A-A makes it possible to couple the first electronic connector 30 to a male connector 41 which is linked via a cable 40 to the physiological sensor 13, the latter producing the incoming analog signal. In this example, the first connector 30 is of data connector type, in particular, it is a medical data connector such as the connectors ODU MINI-MED®, LEMO®, Fisher®, Smith®, Attend®. However, the data connector may be selected from a general type of connector such as a jack socket. The jack socket may make it possible to plug into to the electro-medical device 10 an apparatus such as an electroencephalograph (EEG) or a MEG apparatus which makes it possible to carry out a magnetoencephalography.

According to an embodiment, the electro-medical device 10 may comprise a rechargeable battery 14 arranged in an internal space in the casing 20, for example a specific housing including connector technology allowing it to distribute the electrical power to the electronic means 50 included in the electro-medical device 10. The battery 14 is preferably not removable. To this end, the housing may be sealed.

According to the embodiment illustrated notably in FIG. 8, the electronic means 50 may include a digital converter 51 configured to convert the incoming analog signal 12 into a digital signal 15. Advantageously, the electronic means 50 may also comprise an amplifier. The amplifier advantageously improves the signal to noise ratio of the analog signal 12 before it is converted. The electro-medical device 10 may thus be considered to be a modulator and a casing for relaying physiological data. Moreover, the electronic means 50 may include a microprocessor 52 coupled to a memory 53 for storing the digital signal 15, at least temporarily. The electronic means 50 may comprise several memories such as Eprom, Fram, SD.

As shown in FIG. 8, the electronic means 50 may also comprise a wireless transmitter/receiver 54 configured to transmit the digital signal 15 to the remote terminal 11. The transmitter/receiver 54 may be configured to transmit a near field signal such as NFC, Bluetooth and RFID or a signal of WIFI, Radio, etc. type. Advantageously, the transmitter/receiver 54 selected is of Bluetooth type in order to allow connectivity at a distance of a few meters with the most common connected apparatus such as smartphones, tablets or laptops.

The microprocessor 52 thus commands the transfer of the digital signal 15 which has been converted to a remote terminal 11 or to the memory 53 as shown in FIG. 8. For example, when the electro-medical device 10 is connected to a physiological data sensor 13, and the electro-medical device 10 is not connected to a remote terminal 11, the microprocessor 52 will store the digital signal 15 in the memory 53. The digital signal 15 may thus be stored until the next connection to a remote terminal 11. Since the acquisition of physiological data requires relatively invasive medical procedures, for example a lumbar puncture, it is important that the physiological data measured by the sensor 13 can be retained in the electro-medical device 10 under all circumstances, even when the electro-medical device 10 is not able to transmit the data to a remote terminal 11.

The microprocessor 52 may also be configured to encrypt the wireless signal 16 which is then transmitted to the remote terminal 11. This makes it possible to comply with requirements regarding the confidentiality of personal medical data.

Moreover, the electronic means 50 may comprise an accelerometer configured to switch on the casing and/or pair the transmitter/receiver with a remote terminal. For example, the accelerometer may be configured to wake up the electronic means 50 of the electro-medical device 10 as soon as a certain shaking motion is initiated. According to another possibility which may or may not be associated with the first function of the accelerometer 54, the latter may be configured to pair the transmitter/receiver 54 with a remote terminal following a specific stimulus. The stimulus may for example correspond to two taps on a face of the casing 20.

As can be seen in FIGS. 4 to 7, the electro-medical device 10 may comprise a man/machine interface 56 which is in this case formed on a wall of the casing 20. In this example, the man/machine interface 56 comprises an on/off button 560 and light indicators 561, 562, 563. A first light indicator 561 may inform the user whether the electro-medical device 10 is paired with a remote terminal or in the process of pairing. In this case, the first indicator 561 has the Bluetooth connectivity symbol. A second light indicator 562 may indicate the level of charge or whether the battery is being charged, a battery being depicted to this end. A third indicator 563 may designate the connector 30 to be used to plug in the connector fitting of the sensor 13. Note that the on/off button 560 may also incorporate a light indicator which indicates to the user the state of operation, on or off, of the electro-medical device 10.

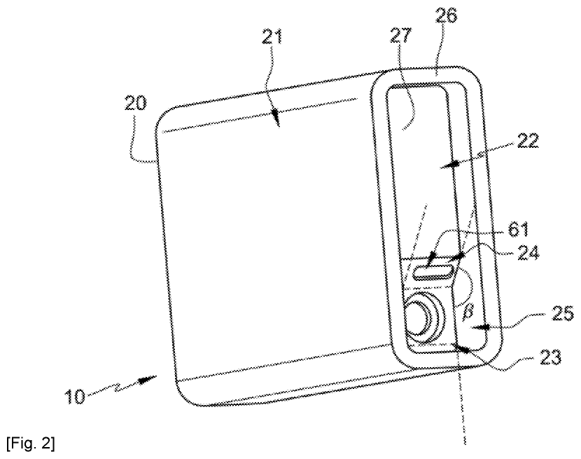

The battery 14 is not visible in FIGS. 1 to 7, but is however shown symbolically in FIG. 8. On the other hand, the casing 20 includes, in the connection zone, a power supply connector 60 which is connected electrically to the battery 14. The connector 60 may also be connected to the electronic components that manage the charging of the battery 14. The power supply connector 60 is visible in FIGS. 1 to 9. In this case, the power supply connector 60 is a female connector which is configured to be connected to a male power supply connector fitting 70 of the same type. The power supply connector 60 makes it possible to transmit an electric current to the battery 14 with a view to charging same. The power supply connector 60 may be selected from connectors such as USB-C, USB-A, DIN, etc. In the example of FIGS. 1 to 7, USB-C is advantageously selected since it offers both powerful transmission and is user friendly since it is reversible, and also because, at the time of drafting of this document, it is very widely used. As shown in FIGS. 2 and 7, the power supply connector 60 comprises a second axis of connection B-B which extends longitudinally along the axis of connection of the male power supply connector fitting 70.

As shown in FIGS. 1 to 8, the first electronic connector 30 and the power supply connector 60 may be arranged close to one another. In practice, the distance between these two connectors 30, 60 may be less than 5 cm, preferably less than 3 cm and more preferably less than 2 cm or even less than 1 cm. The two connectors 30, 60 are thus arranged spatially contiguously in the connection zone of the casing 20. Furthermore, the two connectors 30, 60 may be oriented spatially such that their axes of connection A-A, B-B cross one another. In particular, the two axes of connection A-A, B-B form an angle α of predetermined value as shown in FIGS. 4 and 7. In the example of FIG. 4, the angle α has a value of around 40°, while the example in FIG. 7 shows an angle α the value of which is of the order of 90°. The intersection of the axes of connection A-A, B-B and the spatial proximity of the connectors 30, 60 combine to prevent these two connectors 30, 60 from being coupled at the same time, as shown in FIG. 3. In this figure, the male connector fitting 41 of the data cable 40 is plugged into the first electronic connector 30 along the axis A-A which extends perpendicularly relative to the plane of the wall 22 of the casing 20. As can thus be seen in this FIG. 3, the male connector fitting 41 creates, with the configuration of the connection zone, a mechanical impediment preventing the power supply connector fitting 70 from being plugged into the power supply connector 60. The spatial proximity between the connectors 30, 60 and the intersection of the axes A-A, B-B provide a connection zone which is small and congested. Thus, when a male connector fitting is plugged into one of the connectors 30, 60, it prevents another connector 30, 60 from being coupled at the same time, by virtue of the configuration of the connection zone. As can be seen in FIG. 4, when the power supply connector fitting 70 is plugged into the power supply connector 60, it has the same effect of mechanical impediment. According to this embodiment, the power supply connector fitting 70 intersects the axis A-A at the angle α and clearly prevents the connector fitting compatible with the electronic connector 30 from being plugged in at the same time.

According to the configuration of the connection zone, it is possible to obtain such an effect preventing two simultaneous connections when the value of the angle α is less than 150°, preferably, the value of the angle α is less than 145°, and more preferably, the value of the angle α is less than 140°. The value of the angle α may also be greater than 20°, preferably, the value of the angle α is greater than 30°, more preferably the value of the angle α is greater than or equal to 40°.

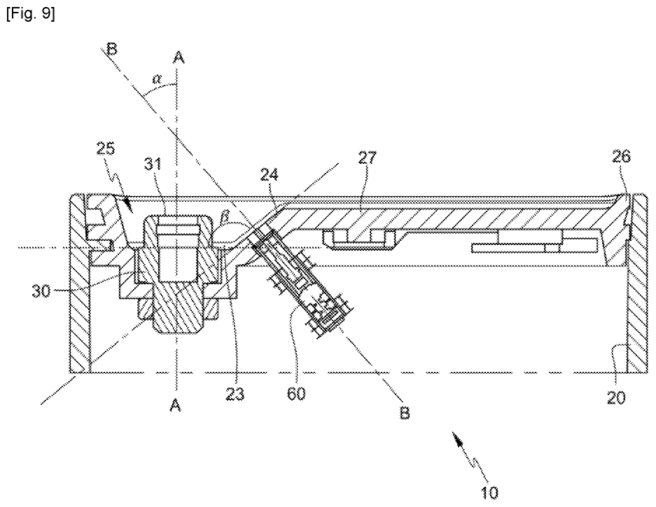

According to an embodiment illustrated in FIGS. 1 to 7, the first electronic connector 30 is integrated in a first plane 23 which is recessed in a wall 22, 22a of the casing 20 while the power supply connector 60 is integrated in a second recessed plane 24 of said wall 22, 22a. The two planes 23, 24 cross one another so that the axes of connection A-A, B-B intersect. The two planes 23, 24 are integrated in the walls 21, 22, 22a of the casing 20 and form the connection zone. The planes 23, 24 may to some extent be considered to be walls of the casing 20, notably in the context of FIGS. 6 and 7. In the example of FIG. 9, it can be seen that the base of the connector 30 comprises a wall which extends in the plane 23.

According to an embodiment illustrated in FIG. 2, the casing 20 includes recessed planes 23, 24 and the respective openings made for the assembly of the connectors 30, 60. In this figure, the recessed planes 23, 24 form an angle β the value of which is greater than 90°. In particular, the value of the angle β is in this case between 160 and 140°. In this embodiment, the inclination of the recessed planes 23, 24 relative to one another provides axes of connection that cross one another as described above.

As shown in FIGS. 1 to 7 and 9, the connectors 30, 60 are arranged in a niche 25 that is set back or recessed relative to a wall 22, 22a of the casing 20. The niche 25 then forms the connection zone of the casing 20. In particular, the second recessed plane 24 creates a break with the plane of a wall 22, 22a of the casing 20. The first recessed plane 23 is set back from the plane of said wall 22, 22a and extends parallel to said wall 22, 22a of the casing 20. The second plane 24 thus constitutes the link between said wall 22, 22a of the casing 20 and the first plane 23.

A first embodiment is illustrated in FIGS. 1 to 4 and 9, wherein the wall 22 on which the connection zone is positioned includes a protruding rim 26 which extends on the periphery of the wall 22. The wall 22 includes a flat area 27 that is set back from the rim 26. In this case, the rim 26 interacts with the recessed planes 23, 24 to form a niche 25 that is more difficult to access, which increases the constraints preventing the two connectors 30, 60 from being coupled at the same time. The flat area 27 also contributes to the mechanical impediment, since the second recessed plane 24 creates a break in the flat area 27 in the vicinity of one end of the wall 22. The end of the wall 22 is located in the vicinity of a corner of the casing 20, the congestion of the connection zone is thus increased by the presence of the rim 26 which surrounds the flat area 27 on three sides at this location.

The flat area 27 may serve for the integration of the man machine interface as shown in FIGS. 3 and 4. The flat area 27 may also be used by the manufacturer to display technical and regulatory information on the electro-medical device 10.

FIG. 9 shows a view in section of the first embodiment. As can be seen, the respective orientations of the axes of connection A-A, B-B are conferred by the integration of each connector 30, 60 in the niche 25 of the casing 20 but also in the circuit board (not visible). It is thus the configuration of the connection zone of the casing 20 which allows the intersection of said axes. In particular, the inclination of the second recessed plane 24 provides an inclined plane in which is formed the opening 61 of the power supply connector 60. As can be seen, the first recessed plane 23 is parallel to the flat area 27 and is sunken with respect to the latter in the niche 25. The second recessed plane 24 thus forms the same angle β with the flat area 27 and the first recessed plane 23. The angle β to some extent defines the inclination of the power supply connector 60 relative to the electronic connector 30 so that the casing 20 has intersecting axes of connection A-A, B-B which prevent these two connectors 30, 60 from being coupled at the same time. Note that the orientations of the connectors 30, 60 are preserved in their integration on the circuit board. As shown in FIG. 9, each connector 30, 60 extends along its respective axis of connection A-A, B-B inside the casing 20, and their orientation is thus retained in their integration on the circuit board.

According to a second embodiment illustrated in FIG. 5, the connection zone may take the form of a U-shaped niche 25 which extends in the lateral wall 22. The niche 25 is arranged on the transverse median axis of the wall 22. In this case, the second recessed plane 24 creates a break from an edge of the wall 22 and is inclined towards the first recessed plane 23. The latter is not visible because the connector 30 protrudes from the first recessed plane 23 and takes up a large part of the available space.

In particular, the connector 30 includes a tube the annular end 31 of which is flush with the plane of the wall 22. In this case, the power supply connector 60 includes an opening 61 which corresponds to the connection port, the opening 61 being arranged under the plane in which the annular end 31 of the connector 30 extends. Furthermore, the opening 61 extends in the second recessed plane 24 which is inclined relative to the annular end 31 which extends in a plane parallel to the first recessed plane 23. This inclination of the opening 61 provides intersecting axes of connection A-A, B-B which do not allow the two connectors 30, 60 to be coupled at the same time, by virtue of the spatial proximity of the connectors 30, 60. Such a configuration is also present in the embodiment illustrated in FIGS. 1 to 4. According to this embodiment, the annular end 31 is substantially in the plane of the flat area 27 of the wall 22. The opening 61 is arranged under the plane of the flat area 27 as can be seen in particular in FIG. 2. In particular, the opening 61 is formed in the second recessed plane 24 and thus has an inclination relative to the connector 30 and its tube.

Note that in FIG. 5, the man/machine interface 56 is formed on the panel wall 21 of the casing 20.

According to a third embodiment illustrated in FIGS. 6 and 7, the second recessed plane 24 is arranged perpendicularly relative, on one side, to the wall 22a, and on another side, to the first recessed plane 23. It can thus be said that the wall 22a has a recess which forms the niche 25 of the connection zone. The niche 25 of the connection zone thus has an L shape or a half U shape, the connector 60 being arranged in the recessed second plane 24 while the connector 30 is arranged in the first recessed plane 23. This arrangement helps to position the two axes of connection A-A, B-B substantially perpendicularly to within a few degrees. In this case, the annular end 31 of the connector 30 is flush with the face of the first recessed plane 23 and the opening 61 of the power supply connector 60 is in a plane above the first recessed plane 23.

Although this spatial organization of the connectors 30, 60 differs from those of other embodiments, the correlation between the spatial proximity of the two connectors 30, 60 and the intersection of the axes of connection A-A, B-B of the connectors 30, 60 prevents the two connectors 30, 60 from being coupled at the same time. As described above, the distance between the two connectors 30, 60 may be less than 5 cm, preferably less than 3 cm and more preferably less than 2 cm, better still the distance may be less than 1 cm.

As shown in FIGS. 6 and 7, the casing 20 may include a second electronic connector 80. In this case, the connector 80 is of jack socket type. However, any type of data connector may be used instead, in particular those used in the field of health and medicine such as ODU MINI-MED®, LEMO®, Fisher®, Smith®, Attend®, etc. As shown in FIGS. 6 and 7, the connector 80 is also contiguous with the power supply connector 60. The spatial proximity between the second electronic connector 80 and the power supply connector 60 is of the same order as between the first electronic connector 30 and the power supply connector 60.

In this example, the connector 80 is adjacent to the connector 30, they are both arranged in the first recessed plane 23. More specifically, the second electronic connector 80 is interposed between the connector 30 and the second recessed plane 24 on which the power supply connector 60 is provided. This configuration gives the connector 80 a third axis of connection C-C which is parallel to the axis of connection A-A of the first electronic connector 30. Consequently, the third axis of connection C-C is perpendicular to the axis of connection B-B of the power supply connector 60. The angle Ω between the axes of connection B-B, C-C is thus identical to the angle α which is formed by the axes of connection A-A, B-B. Again, the spatial proximity between the connectors 60, 80 and the intersection of their axes of connection B-B, C-C prevents these two connectors 60, 80 from being used at the same time.

Conversely, the adjacent position of the electronic connectors 30, 80 in one and the same plane provides axes of connection A-A, C-C that are parallel to one another. Furthermore, the spacing between the two connectors 30, 80 is admittedly small, for example less than 5 cm or even smaller, less than 3 cm and even less than 1 cm, but the fact that they are positioned in the same plane allows these two electronic connectors 30, 80 to be coupled at the same time. Having two identical or different electronic connectors makes it possible to connect the electro-medical device 10 to two separate physiological sensors in such a way as to collect different types of data synchronously.

This may be useful when a practitioner needs to perform complex examinations that require the simultaneous acquisition of types of data such as the intracranial pressure and the cerebral impedance of a patient. Cerebral impedance corresponds to the opposition that the brain tissue produces to the passage of an electric current. Cerebral impedance may be measured using a plurality of techniques such as electroencephalography (EEG) or magnetoencephalography (MEG). Sensors placed on the patient's skull may be used to measure the electrical or electromagnetic signals from the brain.

Simultaneously measuring cerebral impedance and intracranial pressure allows clinicians to achieve a more complete assessment of the neurological state of the patient. This may be useful for monitoring or diagnosing several pathologies or anomalies relating to the brain: hydrocephalus, traumatic brain injury, brain tumors, cerebral edema, brain infections, intracranial hemorrhage, intracranial hypertension, idiopathic intracranial hypertension syndrome, etc. The EEG or EMG sensors may comprise different connector fittings, for example, the EEG sensor generally comprises a male connector fitting of jack type whereas the intracranial pressure sensor may for example be of ODU MINI-MED® type. Thus, the two connectors 30, 80 shown in FIGS. 6 and 7 allow the two simultaneous acquisitions to be received in the electro-medical device 10. The two signals may be processed simultaneously or one after another by the electronic means before being transmitted to the remote terminal 11 as shown in FIG. 8.

Note that the two previous embodiments described in FIGS. 1 to 6 and 9 may also comprise two or even three electronic connectors, to collect data coming from several physiological sensors acquiring data synchronously. To make it possible to include more electronic connectors while retaining the advantages of the invention, the connection zone may be enlarged, in particular the first recessed plane 23 on which the connector 30 is positioned. The additional connector is provided adjacent to the connector 30, in the vicinity of the latter so that the power supply connector 60 cannot be coupled at the same time.

As shown in FIG. 8, according to one embodiment, the invention may also relate to a measurement kit 90 for measuring physiological data of a patient. This measurement kit 90 may be adapted to measure the intracranial pressure or the blood pressure of a patient. To this end, the kit comprises an electro-medical device 10 according to an embodiment of the invention and a physiological sensor 13. Preferably, the physiological sensor 13 is configured to measure the pressure of a physiological fluid of a patient, as explained above. However, the kit may comprise the electro-medical device 10 according to an embodiment of the invention associated with another physiological data sensor such as a pressure sensor.

1. An electro-medical device configured to relay, to a remote terminal, an analog electrical signal representing the physiological data of a patient such as the intracranial pressure or the blood pressure of the patient, the analog electrical signal being produced by an electronic physiological sensor, the electro-medical device comprising:
a casing which includes:
a rechargeable battery arranged in an internal space in the casing and supplying electrical power to the electro-medical device,
a first electronic connector which has a first axis of connection A-A, the first axis of connection A-A making it possible to connect the first electronic connector being configured to be coupled to a connector fitting linked by means of a cable to the electronic physiological sensor and also to receive the incoming analog electrical signal,
a power supply connector which is connected electrically to the battery and is configured to be connected to a power supply connector fitting in order to transmit an electric current to the battery for charging same, the power supply connector comprising a second axis of connection B-B,
wherein the first electronic connector and the power supply connector are arranged spatially contiguously in a connection zone of the casing, and also oriented spatially such that the first axis of connection A-A crosses the second axis of connection B-B, the intersection of the first axis of connection A-A and the second axis of connection B-B, and where the spatial proximity of the first electronic connector and the power supply connector prevent the first electronic connector and the power supply connector from being coupled at the same time.

2. The electro-medical device according to claim 1, wherein, the first electronic connector and the power supply connector are formed in a niche that is set back or recessed relative to a wall of the casing, the niche then forming the connection zone of the casing.

3. The electro-medical device according to claim 1, wherein, the first electronic connector is integrated in a first recessed plane that is recessed relative to a wall of the casing, the power supply connector being integrated in a second recessed plane that is recessed relative to said wall, the first recessed plane and the second recessed plane crossing one another so that the axes of connection A-A and B-B intersect.

4. The electro-medical device according to claim 3, wherein, the second recessed plane creates a break with the plane of said wall of the casing, the first recessed plane being set back from the plane of said wall and extending parallel to said wall, the second recessed plane forming the link between said wall and the first recessed plane.

5. The electro-medical device according to claim 1, wherein, the connection zone is arranged on a wall of the casing which includes a protruding rim, said wall including a flat area that is set back from the protruding rim.

6. The electro-medical device according to claim 4, wherein, the first recessed plane and the second recessed plane form with the protruding rim a niche set back from the flat area.

7. The electro-medical device according to claim 3, wherein, the first recessed plane and the second recessed plane form a shoulder in a wall of the casing.

8. The electro-medical device according to claim 1, wherein, the first electronic connector includes a tube comprising the annular end of which is arranged in a plane which is raised relative to the power supply connector, the power supply connector being inclined relative to the tube.

9. The electro-medical device according to of claim 1, wherein, the first axis of connection A-A and the second axis of connection B-B form an angle α the value of which is less than 150°.

10. The electro-medical device according to claim 1, wherein, the casing includes a second electronic connector contiguous with the power supply connector, the second electronic connector comprising a third axis of connection C-C, the third axis of connection C-C crossing the axis of connection B-B of the power supply connector, the spatial proximity between the power supply connector and the second electronic connector and the intersection of their axes of connection B-B, C-C preventing the second electronic connector and the power supply connector from being coupled at the same time.

11. The electro-medical device according to claim 10, wherein, the second electronic connector is arranged in the same plane as the first electronic connector.

12. The electro-medical device according to claim 1, which includes electronic means comprising:
a digital converter configured to convert the incoming analog electrical signal into a digital signal, and
a remote transmitter/receiver configured to transmit the digital signal to a remote terminal.

13. The electro-medical device according to claim 12, wherein, the electronic means include an internal memory configured to store the converted digital signal.

14. The electro-medical device according to claim 1, which includes a man/machine interface provided on a wall of the casing, the man/machine interface comprising one or more buttons and/or one or more light indicators.

15. A measurement kit for measuring physiological data of a patient such as the intracranial pressure or the blood pressure of a patient, the measurement kit comprising an electro-medical device according to claim 1 and a physiological sensor configured to measure the pressure of a physiological fluid of a patient as well as to produce an analog electrical signal representative of said pressure.
